(19) United States
(12) Patent Application Publication
(10) Pub. No.: US 2021/0085713 A1
(43) Pub. Date: Mar. 25, 2021

(54) COMPOSITIONS AND METHODS FOR TREATING STROKE

(71) Applicants: Michael Levitt, (US); Melanie Walker, (US); University of Virginia Patent Foundation, Charlottesville, VA (US)

(72) Inventors: M. Yashar S. Kalani, Irvine (US); Pedro Norat, Charlottesville, VA (US); Petr Tvrdik, Charlottesville, VA (US); Michael Levitt, Seattle, WA (US); Melanie Walker, Seattle, WA (US)

(73) Assignee: University of Virginia Patent Foundation, Charlottesville, VA (US)

(21) Appl. No.: 17/114,784

(22) Filed: Dec. 8, 2020

Related U.S. Application Data
(63) Continuation of application No. PCT/US2019/036306, filed on Jun. 10, 2019.
(60) Provisional application No. 62/682,259, filed on Jun. 8, 2018.

(51) Int. Cl. A61K 35/12; A61P 43/00

(52) U.S. Cl. CPC A61K 35/12; A61K 9/0085; A61P 43/00

(57) ABSTRACT

The present application provides compositions and methods for treating stroke using mitochondria, precursor cells, and other compounds. It is demonstrated that focused ultrasound can be used as part of the treatment to selectively target the blood brain barrier to enhance the use of the treatments disclosed herein.

### CROSS REFERENCE TO RELATED APPLICATIONS

This application is a continuation of PCT International Patent Application Serial No. PCT/US2019/036306, filed Jun. 10, 2019, herein incorporated by reference in its entirety, which claims benefit of U.S. Provisional Patent Application Ser. No. 62/682,259, filed Jun. 8, 2018, herein incorporated by reference in its entirety.

### BACKGROUND

Stroke is the third leading cause of death and the leading cause of long-term morbidity in the United States (Tymianski, 2013). In 2008, the cost of care for stroke patients amounted to $18.8 billion, and it is estimated that this population had an additional $15.5 billion in loss of productivity. The state of Virginia has an annual age-adjusted stroke rate of 3%, placing it amongst the states most affected by stroke. This means that this year, 199,319 Virginians have had a stroke and are living with the disability caused by this stroke; and, that in the past year nearly 3,300 Virginians have died due to stroke (Virginia Department of Health). For patients suffering from ischemic stroke (85% of all strokes), the only proven efficacious treatments are rapid revascularization using neuroprotective agents, such as tissue plasminogen activator (tPA) (The National Institute of Neurological Disorders and Stroke rt-PA Stroke Study Group, 1995) or mechanical thrombectomy (Berkhemer et al., 2015). Transplantation of progenitor cells has demonstrated some efficacy in a small non-blinded trial in humans (Steinberg et al., 2016) but requires an invasive procedure in a fragile patient population. However, there are currently no medication regimens, biologic regimens, or regenerative regimens to assist with post-stroke recovery.

There is a long felt need in the art for compositions and methods for treating stroke. The presently disclosed subject matter addresses these and other needs in the art.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In some embodiments, provided herein is a method of treating stroke in a subject. In some embodiments, the method comprises administering to the subject an effective amount of a composition comprising mitochondria, precursor cells, or combinations thereof, in a manner in which the composition is delivered to a revascularized bed of tissue in the subject's brain. In some embodiments, the revascularized bed of tissue is provided by opening an occluded blood vessel in the brain of the subject. In some embodiments, the opening of an occluded blood vessel is accomplished by thrombectomy or thrombolysis.

In some embodiments, the mitochondria and/or the precursor cells are autologous to the subject. In some embodiments, the precursor cells are neural precursor cells, mesenchymal precursor cells, or a combination thereof.

In some embodiments, the administering of the composition comprises intra-arterial administration or stereotactic injection. In some embodiments, the intra-arterial administration comprises administration to an internal carotid artery, a vertebral artery, and/or to a branch thereof.

In some embodiments, the composition further comprises a neuroprotective agent. In some embodiments, the composition further comprises a pharmaceutically acceptable carrier. In some embodiments, the pharmaceutically acceptable carrier is pharmaceutically acceptable for use in humans.

In some embodiments, the method comprises opening the blood-brain barrier prior to, during, and/or after the administering of the composition. In some embodiments, opening the blood-brain barrier comprises exposing the subject to focused ultrasound and/or to intra-arterial delivery of mannitol prior to, during, and/or after the administering of the composition.

In some embodiments, provided herein is a pharmaceutical composition for use in treating stroke. In some embodiments, the composition comprises mitochondria, precursor cells, or a combination thereof. In some embodiments, the pharmaceutical composition is adapted for delivery to a revascularized bed of tissue in the subject's brain.

In some embodiments, the mitochondria and/or the precursor cells are autologous to a subject to whom the pharmaceutical composition will be administered. In some embodiments, the precursor cells are neural precursor cells, mesenchymal precursor cells, or a combination thereof.

In some embodiments, the composition is adapted for intra-arterial administration or stereotactic injection. In some embodiments, the composition further comprises a neuroprotective agent. In some embodiments, the composition further comprises a pharmaceutically acceptable carrier. In some embodiments the pharmaceutically acceptable carrier is pharmaceutically acceptable for use in humans.

In some embodiments, a pharmaceutical composition in accordance with the presently disclosed subject matter is provided for use in a method of treating stroke in a subject, the method comprising administering to the subject an effective amount of a composition in a manner in which the composition is delivered to a revascularized bed of tissue in the subject's brain. In some embodiments, the revascularized bed of tissue is provided by opening an occluded blood vessel in the brain of the subject. In some embodiments, the opening of an occluded blood vessel is accomplished by thrombectomy or thrombolysis. In some embodiments, the administering of the composition comprises intra-arterial administration or stereotactic injection with or without focused ultrasound as an adjunct. In some embodiments, the intra-arterial administration comprises administration to an internal carotid artery, a vertebral artery, and/or to a branch thereof.

In some embodiments, a pharmaceutical composition of the presently disclosed subject matter is provided for use in a method of treatment in accordance with the presently disclosed subject matter, wherein the method comprises opening the blood-brain barrier prior to, during, and/or after the administering of the composition. In some embodiments, opening the blood-brain barrier comprises exposing the subject to focused ultrasound and/or to intra-arterial delivery of mannitol prior to, during, and/or after the administering of the composition.

Accordingly, it is an object of the presently disclosed subject matter to provide methods and compositions for treating stroke.

This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description, Drawings and Examples.

### BRIEF DESCRIPTION OF THE DRAWINGS

FIG. 1A is a transmission electron micrograph of isolated mitochondria.

FIG. 1B is a digital image showing mitochondria labelled with MitoTracker Red CMXRos under fluorescence microscopy.

FIG. 1C is a plot showing mitochondrial viability.

FIG. 1D is a plot showing size of the mitochondrial isolates.

FIG. 2 is a schematic demonstrating the timing of ischemia and delivery of mitochondria in a mouse model of stroke.

FIG. 3A is an image showing No Stroke+Mitochondria Intra-arterial, including the following tags DAPI and Mitotracker.

FIG. 3B is a set of images showing Stroke+Mitochondria Intra-arterial, including the following tags DAPI (left image and first upper small panel, right image), CD31 (second upper small panel, right image), NeuN (fourth upper small panel, right image), and Mitotracker (left image and third upper small panel, right image). The lower panel on the right panel of FIG. 3B is a merge image.

FIG. 3C is a set of images showing Stroke+Mitochondria Intra-arterial+FUS, including the following tags DAPI (left image and first upper small panel, right image), CD31 (second upper small panel, right image), NeuN (fourth upper small panel, right image), and Mitotracker (left image and third upper small panel, right image). The lower panel on the right panel of FIG. 3C is a merge image.

FIG. 3D is a set of images showing Stroke+Mitochondria stereotactic injected, including the following tags DAPI (left image and first upper small panel, right image), CD31 (second upper small panel, right image), NeuN (fourth upper small panel, right image), and Mitotracker (left image and third upper small panel, right image). The lower panel on the right panel of FIG. 3D is a merge image.

FIG. 4A is a set of brain images showing No Stroke+Evans Blue dye administered intra-arterially.

FIG. 4B is a set of brain images showing No Stroke+Evans Blue dye administered intra-arterially and with FUS treatment.

FIG. 4C is a set of brain images showing Stroke+Evans Blue dye administered intra-arterially.

FIG. 4D is a set of brain images showing Stroke+Evans Blue dye administered intra-arterially and with FUS treatment.

FIG. 4E is a bar graph showing Evans Blue dye injected intra-arterially.

FIG. 5A is a set of images at 60× magnification showing Stroke+Mitochondria Intra-arterial, including neuron fluorescent staining with the following tags individually DAPI (first panel from left), MAP2 (second panel from left), and Mitotracker (third panel from left), and one merged image (fourth panel from left).

FIG. 5B is a set of images at 60× magnification showing Stroke+Mitochondria Intra-arterial, including microglia fluorescent staining with the following tags individually DAPI (first panel from left), Iba-1 (second panel from left), and Mitotracker (third panel from left), and one merged image (fourth panel from left).

FIG. 5C is a set of images at 60× magnification showing Stroke+Mitochondria Intra-arterial, including astrocyte fluorescent staining with the following tags individually DAPI (first panel from left), GFAP (second panel from left), and Mitotracker (third panel from left), and one merged image (fourth panel from left).

FIG. 6A is a set of images at 60× magnification showing Stroke+Mitochondria Intra-arterial+FUS, including neuron fluorescent staining with the following tags individually DAPI (first panel from left), MAP2 (second panel from left), and Mitotracker (third panel from left), and one merged image (fourth panel from left).

FIG. 6B is a set of images at 60× magnification showing Stroke+Mitochondria Intra-arterial+FUS, including microglia fluorescent staining with the following tags individually DAPI (first panel from left), Iba-1 (second panel from left), and Mitotracker (third panel from left), and one merged image (fourth panel from left).

FIG. 6C is a set of images at 60× magnification showing Stroke+Mitochondria Intra-arterial+FUS, including astrocyte fluorescent staining with the following tags individually DAPI (first panel from left), GFAP (second panel from left), and Mitotracker (third panel from left), and one merged image (fourth panel from left).

FIG. 7A is a set of images at 60× magnification showing Stroke+Mitochondria Stereotactic, including neuron fluorescent staining with the following tags individually DAPI (first panel from left), MAP2 (second panel from left), and Mitotracker (third panel from left), and one merged image (fourth panel from left).

FIG. 7B is a set of images at 60× magnification showing Stroke+Mitochondria Stereotactic, including microglia fluorescent staining with the following tags individually DAPI (first panel from left), MAP2 (second panel from left), and Mitotracker (third panel from left), and one merged image (fourth panel from left).

FIG. 7C is a set of images at 60× magnification showing Stroke+Mitochondria Stereotactic, including astrocyte fluorescent staining with the following tags individually DAPI (first panel from left), MAP2 (second panel from left), and Mitotracker (third panel from left), and one merged image (fourth panel from left).

FIG. 8A is an image showing Stroke+DSRed Mitochondria Intra-arterial, including fluorescent staining with the following tags individually DAPI and DSRed. Scale Bar=5 μm.

FIG. 8B is an image showing Stroke+DSRed Mitochondria Intra-arterial+FUS, including fluorescent staining with the following tags individually DAPI and DSRed. Scale Bar=5 μm.

FIG. 8C is a set of images at 60× magnification showing Stroke+DSRed Mitochondria Intra-arterial, including fluorescent staining with the following tags individually DAPI (first panel from left), GFAP (second panel from left), MAP2 (third panel from left), Mitotracker (fourth panel from left), and one merged image (fifth panel from left). Scale Bar=5 μm.

FIG. 8D is a set of images at 60× magnification showing Stroke+DSRed Mitochondria Intra-arterial+FUS, including fluorescent staining with the following tags individually DAPI (first panel from left), GFAP (second panel from left), MAP2 (third panel from left), Mitotracker (fourth panel from left), and one merged image (fifth panel from left). Scale Bar=5 μm.

FIGS. 9A-9C are sets of images showing that high-frequency focused ultrasound does not result in haemorrhage, both based on MRI and histology (FIG. 9A and FIG. 9C). Higher frequency levels and use of anionic microbubbles can result in haemorrhage (FIG. 9B).

FIG. 10 is a graph showing an ATP assay in the stroked hemispheres after mitochondria delivery.

FIG. 11A is a set of brain images showing Stroke+Vehicle administered intra-arterially.

FIG. 11B is a set of brain images showing Stroke+Mitochondria administered intra-arterially.

FIG. 11C is a plot showing distal MCA occlusion after mitochondria re injected intra-arterially.

FIGS. 12A and 12B are a plot and images, respectfully, showing flow cytometry on hemispheres that received mitochondria and controls.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

Mitochondria are fundamental for metabolic homeostasis in all multicellular eukaryotes. In the nervous system, mitochondria-generated adenosine triphosphate (ATP) is required to establish appropriate electrochemical gradients and reliable synaptic transmission. In some embodiments, the presently disclosed subject matter demonstrates that autologously harvested mitochondria can be delivered intra-arterially after ischemia to the brain; they traverse the blood brain barrier; they are engulfed by cells of the central nervous system; and they function as a neuroprotectant in this setting. In some embodiments, high-frequency focused ultrasound (FUS) is used in the delivery of mitochondria past the blood brain barrier. The presently disclosed results are the first to demonstrate a neuroprotectant role for autologously harvested and intra-arterially delivered mitochondria. These results have immediate clinical translatability, given results of recent thrombectomy trials in patients.

In some embodiments, the presently disclosed subject matter involves the use of autologously harvested mitochondria as neuroprotectants after cerebral ischemia. Mitochondria were isolated and delivered using stereotactic delivery versus intra-arterial delivery with and without the use of FUS to augment blood brain barrier opening. Isolation of biochemically active mitochondria can be readily achieved in the laboratory and clinical setting. Preble et al., J Vis Exp. Epub 2014 Sep. 6.:e51682; McCully et al., Clin Transl Med. Springer; 2016; 5:16. Some benefits of this method for isolating mitochondria are the feasible time of the isolation process (up to 30 minutes), the use of autologous tissue of the patient with a simple biopsy needle, and the viability and number of mitochondria isolated. These features make mitochondria a favorable candidate cell/organelle for study in clinical trials for a wide array of neurological disorders, including but not limited to after large vessel occlusion where the mitochondria can be delivered, for example intra-arterially delivered, into a revascularized ischemic bed.

Based on the examples disclosed herein, the present application provides compositions and methods useful for treating stroke. The present application provides compositions and methods for improving post-stroke recovery.

Various aspects and embodiments of the presently disclosed subject matter are described in further detail below.

### EMBODIMENTS

In accordance with the presently disclosed subject matter, delivery, such as intra-arterial delivery, of mitochondria into a revascularized bed of tissue, such as in the brain, with or without the use of focused ultrasound or other chemical or physical approaches of opening the blood brain barrier, provides neuroprotection and a decrease in the size of stroke. Disclosed herein are unexpected results using a model of temporary middle cerebral artery occlusion in mice where it is demonstrated that administration of intra-carotid mitochondria after opening of the blood brain barrier using focused ultrasound results in a decrease in the volume of infarcted brain. Thus, in some embodiments, the presently disclosed subject matter provides for the delivery of mitochondria into tissue. Unexpectedly, the compositions and methods are useful for delivering mitochondria into brain tissue. The presently disclosed subject matter further provides compositions and methods for treatment of stroke using focused ultrasound to selectively open the blood brain barrier to allow for selective targeting when using a treatment regimen as disclosed herein.

In some embodiments, the presently disclosed subject matter provides for a revascularized bed of tissue in the brain of a subject that suffered a stroke by opening an occluded blood vessel in the subject using chemical or mechanical thrombectomy. Following this procedure, with a microcatheter or a guide catheter immediately adjacent to the compromised vascular bed, mitochondria are delivered to function as a neuroprotectant. In some embodiments, the addition of the focused ultrasound or intra-arterial mannitol serves to enhance opening of the blood brain barrier.

The presently disclosed subject matter further provides for the use of cells, particularly precursor cells, such as stem cells. In some embodiments, the presently disclosed subject matter provides for the use of neural precursor cells or mesenchymal precursor cells, such as neural stem cells or mesenchymal stem cells. The United States Food and Drug Administration (FDA) has an exemption for “cell-based therapies”, where the cell source is from the patient and the cells are harvested and processed during the same operative procedure. The presently disclosed subject matter encompasses these procedures for treating patients. In some aspects, the cell is a human cell.

Thus, in accordance with some embodiments of the presently disclosed subject matter a method of treating stroke in a subject is provided. In some embodiments, the method comprising administering to the subject an effective amount of a composition comprising mitochondria, precursor cells, or combinations thereof, in a manner in which the composition is delivered to a revascularized bed of tissue in the subject's brain. In some embodiments, the revascularized bed of tissue is provided by opening an occluded blood vessel in the brain of a subject that suffered a stroke. The opening of the occluded blood vessel increases and/or improves blood flow (in some embodiments, to a normal or basal blood flow) to the tissue and/or inhibits decreased blood flow to the tissue. The opening of an occluded blood vessel can be accomplished by thrombectomy or thrombolysis for stroke using an intra-arterial route (such as with a microcatheter or a guide catheter) with or without the use of focused ultrasound. Mechanical thrombectomy can carried out using any suitable device as would be apparent to one of ordinary skill in the art upon a review of the instant disclosure, typically with the device or component thereof immediately adjacent to the compromised vascular bed. Representative such devices include coil retrievers, aspiration devices, and stent retrievers. Representative such devices are also disclosed in U.S. Pat. Nos. 10,271,864; 10,010,335; and 9,962,178, each of which is hereby incorporated by reference in its entirety. Chemical thrombectomy, or thrombolysis, can be accomplishing using any suitable neuroprotective agent, such as tissue plasminogen activator (tPA). Subjects suffering from ischemic stroke or hemorrhagic stroke can be treated.

In some embodiments, the mitochondria and/or the precursor cells are autologous to the subject. In some embodiments, the precursor cells are neural precursor cells or mesenchymal precursor cells. Approaches for isolating and/or preparing mitochondria and precursor cells are disclosed elsewhere herein and are known in the art, as would be appreciated by one of ordinary skill in the art upon a review of the instant disclosure.

In some embodiments, the administering of the composition comprises intra-arterial administration or stereotactic injection, with or without focused ultrasound as an adjunct. In some embodiments, the intra-arterial administration comprises administration to an internal carotid artery or branch thereof of, and/or a vertebral artery or branch thereof. In some embodiments, the intra-arterial route employs a microcatheter or a guide catheter that used in generating a revascularized bed of tissue.

In some embodiments, the composition further comprises a neuroprotective agent, such as tissue plasminogen activator (tPA). In some embodiments, the composition further comprises a pharmaceutically acceptable carrier. In some embodiments, the pharmaceutically acceptable carrier is pharmaceutically acceptable for use in humans. In some embodiments, the composition comprises an additional therapeutic agent. Such agents include but, are not limited to, amino acids, antisense oligonucleotides, antibodies, siRNA, and the like, as are described in the definitions set forth herein below.

In some embodiments, the method comprises comprising opening, for example selectively and/or transiently opening, the blood-brain barrier prior to, during, and/or after the administering of the composition. Representative approaches for opening the blood brain barrier include focused ultrasound (FUS) and intra-arterial delivery of mannitol. Thus, in some embodiments, the presently disclosed subject matter uses focused ultrasound to selectively open the blood brain barrier to allow for selective targeting when using a treatment regimen as disclosed herein. Representative approaches for opening (e.g. transiently opening) the blood-brain barrier are also disclosed in the following patent documents: Published US Patent Application No. US20170259086A1; Published US Patent Application No. US20130006106A1; and U.S. Pat. No. 9,221,867 B2, each of which is incorporated herein by reference in its entirety.

In some embodiments, a combination treatment is used to treat a subject in need thereof. In some aspects, a subject in need thereof is treated with a regimen selected from the group consisting of mitochondria delivery, precursor cell delivery, neuroprotective agent administration, and combinations thereof. In one aspect, two or more of these regimens are used. In one aspect, the precursor cells are neural precursor cells, mesenchymal precursor cells, or combinations thereof. In one aspect, the precursor cells are autologous. In one aspect, the mitochondria are autologous. In one embodiment, focused ultrasound is also used for selective targeting and opening of the blood brain barrier in conjunction with the use of mitochondria, precursor cells, and/or neuroprotective drugs or additional therapeutic agents.

In some embodiments, a pharmaceutical composition comprising mitochondria, precursor cells, neuroprotective agents, or a combination thereof is provided and is administered to a subject in need thereof. In some embodiments, the pharmaceutical composition further comprises a pharmaceutically acceptable carrier. In some embodiments, the pharmaceutically acceptable carrier is pharmaceutically acceptable for use in humans. In some embodiments, the pharmaceutical composition comprises an additional therapeutic agent. Such agents include but are not limited to amino acids, an antimicrobial agent, antisense oligonucleotides, antibodies, siRNA, and the like, as are described in the definitions set forth herein below.

In some aspects, the administration of the pharmaceutical composition is performed in conjunction with focused ultrasound. In some embodiments, the mitochondria and/or the precursor cells are autologous to a subject to whom the pharmaceutical composition will be administered. In some embodiments, the precursor cells are neural precursor cells or mesenchymal precursor cells.

In some embodiments, the pharmaceutical composition is adapted for intra-arterial administration or stereotactic injection. In some embodiments, the intra-arterial administration comprises administration to an internal carotid artery or branch thereof and/or to a vertebral artery or branch thereof.

Based on the disclosure provided herein, one of ordinary skill in the art can determine the dosage of cells or mitochondria for delivery and whether to use focused ultrasound and additional therapeutic agents such as neuroprotective agents. By way of example and not limitation, for mitochondria dosage can be based on biological activity such as the ATP assay; and for cells dosage can be based on cell count and viability.

Given the impact of ischemic stroke on the population of the United States and the lack of availability of agents that can assist with post-stroke recovery, a therapeutic regimen targeting this patient population is greatly needed. Systemic delivery of progenitor cells or mitochondria to this fragile patient population in a non-invasive manner, perhaps in the rehabilitation setting, in accordance with the presently disclosed subject matter addresses this need. Therapies that can aid improve patient outcomes after stroke have the potential to improve patient quality of life and decrease costs to the health-care system and the families of patients afflicted by stroke.

In some embodiments, the presently disclosed subject matter provides for methods and compositions for use in treating injuries, wounds, diseases, and/or disorders. A subject having a site of injury or wound, or in some cases a disease or disorder, may be susceptible to decreased blood flow at that site and therefore be in need of treatment. In one aspect, the subject had a stroke. In one aspect, the decreased blood flow is in microvessels. These conditions may typically arise from many types of injury including trauma, surgery, and trauma to the skin and/or exposed soft tissue, resulting in an inflammatory reaction and decreased blood flow, particularly in the microvasculature. The types of injuries, disease, and disorders encompassed by the presently disclosed subject matter therefore include, bone trauma, diseases, and disorders, burns, chronic wounds, and surgical procedures such as microvascular surgery, skin flaps and skin grafts, and tissue injury resulting from, for example, a burn, scrape, cut, incision, laceration, ulcer, body piercing, bite wound, trauma, stab wound, gunshot wound, surgical wound, stretch injury, crush wound, compression wound, fracture, sprain, strain, stroke, infarction, aneurysm, herniation, ischemia, fistula, dislocation, radiation, cell, tissue or organ grafting and transplantation, injuries sustained during medical procedures, or cancer. Such injuries include, but are not limited to, bone injury, skin injury, muscle injury, brain injury, eye injury, or spinal cord injury. Tissue injury can include joint injury, back injury, heart injury, vascular system injury, soft tissue injury, cartilage injury, lymphatic system injury, tendon injury, ligament injury, or abdominal injury.

While it is important to treat any condition in which the potential for cell or tissue damage exists immediately (e.g., an acute wound), it is essential that certain conditions be treated before they become chronic conditions. Chronic diseases are a challenge to the patient, the health care professional, and to the health care system. They significantly impair the quality of life for millions of people in the United States. Intensive treatment is required with a high cost to society in terms of lost productivity and health care dollars. The management of chronic diseases can place an enormous strain on health care resources. Diseases or conditions, for example, wounds that were once acute but have progressed to chronic often do so because the diseases cannot be controlled or treated with known therapies. Therefore, there is a need for improved therapies for treating chronic diseases and conditions characterized by cell and tissue damage.

In some embodiments, the presently disclosed subject matter is useful for delivering mitochondria, precursor cells and other agents to neural tissue as well as to other types of tissue. Additionally, the addition of the focused ultrasound or intra-arterial mannitol serves to enhance opening of the blood brain barrier, which is useful for other applications, such as but not limited to the delivery of chemotherapy.

The presently disclosed subject matter further provides for the use of cells, particularly precursor cells, such as stem cells. In some aspects, a cell type useful for treatment, includes, but is not limited to, a cell selected from the group consisting of stem cells, pluripotent stem cells, committed stem cells, embryonic stem cells, adult stem cells, bone marrow stem cells, bone marrow-derived stem cells, adipose stem cells, mesenchymal stem cells, umbilical cord stem cells, dura mater stem cells, differentiated cells, osteoblasts, osteoclasts, myoblasts, neuroblasts, fibroblasts, glioblasts, germ cells, hepatocytes, chondrocytes, keratinocytes, smooth muscle cells, cardiac muscle cells, connective tissue cells, glial cells, epithelial cells, endothelial cells, hormone-secreting cells, cells of the immune system, normal cells, cancer cells, Schwann cells, and neurons. In one aspect, the cell is a human cell.

The presently disclosed subject matter further provides compositions and methods for delivering a cell, material or compound to a subject in need thereof.

The presently disclosed subject matter further provides a method for delivering one or more substances from the group consisting of cells, precursor cells, genes, drugs, proteins, chemicals, bioactive molecules, growth factors, and therapeutic proteins and peptides.

### Definitions

The terminology used herein is for the purpose of describing particular embodiments only and is not intended to be limiting of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

While the following terms are believed to be well understood by one of ordinary skill in the art, the following definitions are set forth to facilitate explanation of the presently disclosed subject matter.

The articles “a” and “an” are used herein to refer to one or to more than one (i.e., to at least one) of the grammatical object of the article. By way of example, “an element” means one element or more than one element. The singular forms “a,” “an,” and “the” include plural reference unless the context clearly dictates otherwise.

The term “abluminal” refers to something being directed away from the lumen of a tubular structure, i.e., a blood vessel.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The terms “additional therapeutically active compound” or “additional therapeutic agent”, as used in the context of the present invention, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated. The additional compounds may also be used to treat symptoms associated with the injury, disease or disorder, including, but not limited to, pain and inflammation.

The term “adult” as used herein, is meant to refer to any non-embryonic or nonjuvenile subject.

A disease or disorder is “alleviated” if the severity of a symptom of the disease, condition, or disorder, or the frequency with which such a symptom is experienced by a subject, or both, are reduced.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

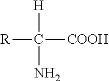

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino-and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies.

The term “antimicrobial agents” as used herein refers to any naturally-occurring, synthetic, or semi-synthetic compound or composition or mixture thereof, which is safe for human or animal use as practiced in the methods of this invention, and is effective in killing or substantially inhibiting the growth of microbes. “Antimicrobial” as used herein, includes antibacterial, antifungal, and antiviral agents.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

The term “autologous”, as used herein, refers to something that occurs naturally and normally in a certain type of tissue or in a specific structure of the body. In transplantation, it refers to a graft in which the donor and recipient areas are in the same individual, or to blood that the donor has previously donated and then receives back, usually during surgery.

The term “basal medium”, as used herein, refers to a minimum essential type of medium, such as Dulbecco's Modified Eagle's Medium, Ham's F12, Eagle's Medium, RPMI, AR8, etc., to which other ingredients may be added. The term does not exclude media which have been prepared or are intended for specific uses, but which upon modification can be used for other cell types, etc.

The term “biocompatible,” as used herein, refers to a material that does not elicit a substantial detrimental response in the host.

The term “biodegradable,” as used herein, means capable of being biologically decomposed. A biodegradable material differs from a non-biodegradable material in that a biodegradable material can be biologically decomposed into units which may be either removed from the biological system and/or chemically incorporated into the biological system.

The term “biological sample,” as used herein, refers to samples obtained from a living organism, including skin, hair, tissue, blood, plasma, cells, sweat, and urine.

The term “bioresorbable,” as used herein, refers to the ability of a material to be resorbed in vivo. “Full” resorption means that no significant extracellular fragments remain. The resorption process involves elimination of the original implant materials through the action of body fluids, enzymes, or cells. Resorbed calcium carbonate may, for example, be redeposited as bone mineral, or by being otherwise re-utilized within the body, or excreted. “Strongly bioresorbable,” as the term is used herein, means that at least 80% of the total mass of material implanted is resorbed within one year.

The phrases “cell culture medium,” “culture medium” (plural “media” in each case) and “medium formulation” refer to a nutritive solution for cultivating cells and may be used interchangeably.

The term “clearance”, as used herein refers to the physiological process of removing a compound or molecule, such as by diffusion, exfoliation, removal via the bloodstream, and excretion in urine, or via sweat or other fluid.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell, tissue, sample, or subject is one being examined or treated.

A “pathoindicative” cell, tissue, or sample is one which, when present, is an indication that the animal in which the cell, tissue, or sample is located (or from which the tissue was obtained) is afflicted with a disease or disorder. By way of example, the presence of one or more breast cells in a lung tissue of an animal is an indication that the animal is afflicted with metastatic breast cancer.

A tissue “normally comprises” a cell if one or more of the cell are present in the tissue in an animal not afflicted with a disease or disorder.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

A “compound,” as used herein, refers to any type of substance or agent that is commonly considered a drug, or a candidate for use as a drug, combinations, and mixtures of the above, as well as polypeptides and antibodies of the invention.

“Cytokine”, as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets, and effector activities of these cytokines have been described.

The term “decreased blood flow”, as used herein, refers to a decrease in blood flow at a site of injury, disease, or disorder, and includes, but is not limited, a decrease in flow rate, an increase in stasis, and an increase in sludging in the vessels.

The term “delivery vehicle” refers to any kind of device or material, which can be used to deliver cells in vivo or can be added to a composition comprising cells administered to an animal. This includes, but is not limited to, implantable devices, aggregates of cells, matrix materials, gels, etc.

As used herein, a “derivative” of a compound refers to a chemical compound that may be produced from another compound of similar structure in one or more steps, as in replacement of H by an alkyl, acyl, or amino group.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” means an amount sufficient to produce a selected effect.

The term “feeder cells” as used herein refers to cells of one type that are co-cultured with cells of a second type, to provide an environment in which the cells of the second type can be maintained, and perhaps proliferate. The feeder cells can be from a different species than the cells they are supporting. Feeder cells can be non-lethally irradiated or treated to prevent their proliferation prior to being co-cultured to ensure to that they do not proliferate and mingle with the cells which they are feeding. The terms, “feeder cells”, feeders,” and “feeder layers” are used interchangeably herein.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, a “functional” molecule is a molecule in a form in which it exhibits a property or activity by which it is characterized.

“Graft” refers to any free (unattached) cell, tissue, or organ for transplantation.

“Allograft” refers to a transplanted cell, tissue, or organ derived from a different animal of the same species.

“Xenograft” refers to a transplanted cell, tissue, or organ derived from an animal of a different species.

The term “growth factor” as used herein means a bioactive molecule that promotes the proliferation of a cell or tissue. Growth factors useful in the present invention include, but are not limited to, transforming growth factor-alpha (TGF-α), transforming growth factor-beta (TGF-β), platelet-derived growth factors including the AA, AB and BB isoforms (PDGF), fibroblast growth factors (FGF), including FGF acidic isoforms 1 and 2, FGF basic form 2, and FGF 4, 8, 9 and 10, nerve growth factors (NGF) including NGF 2.5s, NGF 7.0s and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), EG-VEGF, VEGF-related protein, Bv8, VEGF-E, granulocyte colony stimulating factor (G-CSF), insulin like growth factor (IGF) I and II, hepatocyte growth factor, glial neurotrophic growth factor, stem cell factor (SCF), keratinocyte growth factor (KGF), skeletal growth factor, bone matrix derived growth factors, and bone derived growth factors and mixtures thereof. Some growth factors may also promote differentiation of a cell or tissue. TGF, for example, may promote growth and/or differentiation of a cell or tissue.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

The term “improved blood flow,” as used herein, refers to increased blood flow in a subject being treated according to the presently disclosed subject matter compared with the flow in a subject with an otherwise identical injury or condition not being treated according to the methods of the invention. Improved flow can include less stasis, less sludging, or a combination of both, in the subject being treated compared with the untreated subject. One of ordinary skill in the art will appreciate that there are multiple parameters which can be used as measures or signs of increased blood flow, as well as multiple techniques to determine increased blood flow.

The term “ingredient” refers to any compound, whether of chemical or biological origin, that can be used in cell culture media to maintain or promote the proliferation, survival, or differentiation of cells. The terms “component,” “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art, in accordance with the particular need.

The term “inhibit”, as used herein, refers to the ability of a compound, agent, or method to reduce or impede a described function, level, activity, rate, etc., based on the context in which the term “inhibit” is used. Preferably, inhibition is by at least 10%, more preferably by at least 25%, even more preferably by at least 50%, and most preferably, the function is inhibited by at least 75%. The term “inhibit” is used interchangeably with “reduce” and “block”.

“Inhibiting decreased blood flow” as described herein, refers to any method or technique which inhibits the decrease in blood flow or associated changes in blood flow following injury or stroke, or where decreased blood flow is associated with a disease or disorder. Inhibition can be direct or indirect. One of ordinary skill in the art will appreciate that there are multiple parameters which can be used as measures or signs of blood flow, as well as multiple techniques to determine blood flow.

The term “inhibitor” as used herein, refers to any compound or agent, the application of which results in the inhibition of a process or function of interest, including, but not limited to, differentiation and activity. Inhibition can be inferred if there is a reduction in the activity or function of interest.

As used herein “injecting or applying” includes administration of a compound of the invention by any number of routes and approaches including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, or rectal approaches.

As used herein, “injury” generally refers to damage, harm, or hurt; usually applied to damage inflicted on the body by an external force.

As used herein, an “instructional material” includes a publication, a recording, a diagram, or any other medium of expression, which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container, which contains the identified compound invention, or be shipped together with a container, which contains the identified compound. Alternatively, the instructional material may be shipped separately from the container with the intention that the instructional material and the compound be used cooperatively by the recipient.

Used interchangeably herein are the terms “isolate” and “select”.

The term “isolated”, when used in reference to cells, refers to a single cell of interest, or population of cells of interest, at least partially isolated from other cell types or other cellular material with which it naturally occurs in the tissue of origin (e.g., adipose tissue). A sample of precursor cells is “substantially pure” when it is at least 60%, or at least 75%, or at least 90%, and, in certain cases, at least 99% free of cells other than cells of interest. Purity can be measured by any appropriate method, for example, by fluorescence-activated cell sorting (FACS), or other assays, which distinguish cell types.

An “isolated nucleic acid” refers to a nucleic acid segment or fragment, which has been separated from sequences, which flank it in a naturally occurring state, e.g., a DNA fragment that has been removed from the sequences, which are normally adjacent to the fragment, e.g., the sequences adjacent to the fragment in a genome in which it naturally occurs. The term also applies to nucleic acids, which have been substantially purified, from other components, which naturally accompany the nucleic acid, e.g., RNA or DNA, or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically binds to a target compound. A ligand (e.g., an antibody) “specifically binds to” or “is specifically immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow and Lane, 1988, Antibodies, a Laboratory Manual, Cold Spring Harbor Publications, New York, for a description of immunoassay formats and conditions that can be used to determine specific immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to either a molecule that joins two other molecules covalently or noncovalently, e.g., through ionic or hydrogen bonds or van der Waals interactions.

The term “modulate”, as used herein, refers to changing the level of an activity, function, or process. The term “modulate” encompasses both inhibiting and stimulating an activity, function, or process. The term “modulate” is used interchangeably with the term “regulate” herein.

The term “neuroprotective agent” is mean to refer to a composition, drug, or other agent intended to reverse or prevent damage to the brain, spinal cord, or other neural tissue from ischemia, stroke, convulsions, trauma, or other disease or disorder. Such agents can be administered before and/or after the event, and act by a range of mechanisms. In embodiments, a neuroprotective agent has a clot-busting activity. A representative neuroprotective agent is tissue plasminogen activator (tPA).

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically acceptable carrier” means a chemical composition with which an appropriate compound or derivative can be combined and which, following the combination, can be used to administer the appropriate compound to a subject. In some embodiments, the subject is a human and thus, the pharmaceutically acceptable carrier is pharmaceutically acceptable for use in humans.

As used herein, the term “physiologically acceptable” ester or salt means an ester or salt form of the active ingredient which is compatible with any other ingredients of the pharmaceutical composition, which is not deleterious to the subject to which the composition is to be administered.

The term “prevent,” as used herein, means to stop something from happening, or taking advance measures against something possible or probable from happening. In the context of medicine, “prevention” generally refers to action taken to decrease the chance of getting a disease or condition.

The term “progeny” of a precursor cell as used herein refers to a cell which is derived from a precursor cell and may still have all of the differentiation abilities of the parental precursor cell, i.e., multipotency, or one that may no longer be multipotent, but is now committed to being able to differentiate into only one cell type, i.e., a committed cell type. The term may also refer to a differentiated cell.

A “prophylactic” treatment is a treatment administered to a subject who does not exhibit signs of a disease or injury or exhibits only early signs of the disease or injury for the purpose of decreasing the risk of developing pathology associated with the disease or injury.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. A “significant detectable level” is an amount of contaminate that would be visible in the presented data and would need to be addressed/explained during analysis of the forensic evidence.

A “reversibly implantable” device is one which may be inserted (e.g. surgically or by insertion into a natural orifice of the animal) into the body of an animal and thereafter removed without great harm to the health of the animal.

A “sample,” as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

As used herein, “scaffold” refers to a supporting framework, such as one for bone or tissue growth, either in vivo or in vitro.

As used herein, the term “secondary antibody” refers to an antibody that binds to the constant region of another antibody (the primary antibody).

The terms “solid support”, “surface” and “substrate” are used interchangeably and refer to a structural unit of any size, where said structural unit or substrate has a surface suitable for immobilization of molecular structure or modification of said structure and said substrate is made of a material such as, but not limited to, metal, metal films, glass, fused silica, synthetic polymers, and membranes.

By “small interfering RNAs (siRNAs)” is meant, inter alia, an isolated dsRNA molecule comprised of both a sense and an anti-sense strand. In one aspect, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

By the term “specifically binds,” as used herein, is meant a molecule which recognizes and binds a specific molecule, but does not substantially recognize or bind other molecules in a sample, or it means binding between two or more molecules as in part of a cellular regulatory process, where said molecules do not substantially recognize or bind other molecules in a sample.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. “Standard” can also refer to an “internal standard”, such as an agent or compound which is added at known amounts to a sample and which is useful in determining such things as purification or recovery rates when a sample is processed or subjected to purification or extraction procedures before a marker of interest is measured. Internal standards are often but are not limited to, a purified marker of interest which has been labeled, such as with a radioactive isotope, allowing it to be distinguished from an endogenous substance in a sample.

The term “stimulate” as used herein, means to induce or increase an activity or function level such that it is higher relative to a control value. The stimulation can be via direct or indirect mechanisms. In one aspect, the activity or function is stimulated by at least 10% compared to a control value, more preferably by at least 25%, and even more preferably by at least 50%. The term “stimulator” as used herein, refers to any composition, compound or agent, the application of which results in the stimulation of a process or function of interest, including, but not limited to, wound healing, angiogenesis, bone healing, osteoblast production and function, and osteoclast production, differentiation, and activity.

A “subject” of diagnosis or treatment is an animal, including a human. It also includes pets and livestock.

As used herein, a “subject in need thereof” is a patient, animal, mammal, or human, who will benefit from the method of this invention.

A “surface active agent” or “surfactant” is a substance that has the ability to reduce the surface tension of materials and enable penetration into and through materials.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The term “thermal injury” is used interchangeably with “thermal burn” herein.

“Tissue” means (1) a group of similar cells united to perform a specific function; (2) a part of an organism consisting of an aggregate of cells having a similar structure and function; or (3) a grouping of cells that are similarly characterized by their structure and function, such as muscle or nerve tissue.

The term “tissue injury-associated decreased blood flow”, as used herein, refers to the decrease in blood flow which occurs following an injury, such as a wound, a fracture, a surgical procedure, or a thermal injury. The decrease in blood flow includes, but is not limited to, decreased volume, rate, stasis, or sludging. One of ordinary skill in the art will appreciate that there are multiple parameters which can be used as measures or signs of decreased blood flow, as well as multiple techniques to determine decreased blood flow.

The term “topical application,” as used herein, refers to administration to a surface, such as the skin. This term is used interchangeably with “cutaneous application” in the case of skin. A “topical application” is a “direct application”.

By “transdermal” delivery is meant delivery by passage of a drug through the skin or mucosal tissue and into the bloodstream. Transdermal also refers to the skin as a portal for the administration of drugs or compounds by topical application of the drug or compound thereto. “Transdermal” is used interchangeably with “percutaneous.”

As used herein, the term “treating” may include prophylaxis of the specific injury, disease, disorder, or condition, or alleviation of the symptoms associated with a specific injury, disease, disorder, or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease. “Treating” is used interchangeably with “treatment” herein. In the context of stroke, treating in accordance with the presently disclosed subject matter includes providing a decrease in the volume of infarcted brain.

As used herein “wound” or “wounds” may refer to any detectable break in the tissues of the body, such as injury to skin or to an injury or damage, or to a damaged site associated with a disease or disorder. As used herein, the term “wound” relates to a physical tear, break, or rupture to a tissue or cell layer. A wound may occur by any physical insult, including a surgical procedure or as a result of a disease, disorder condition. Although the terms “wound” and “injury” are not always defined exactly the same way, the use of one term herein, such as “injury”, is not meant to exclude the meaning of the other term.

### Chemical Definitions

As used herein, the term “halogen” or “halo” includes bromo, chloro, fluoro, and iodo.

The term “haloalkyl” as used herein refers to an alkyl radical bearing at least one halogen substituent, for example, chloromethyl, fluoroethyl or trifluoromethyl and the like.

The term “C₁-Cₙ alkyl” wherein n is an integer, as used herein, represents a branched or linear alkyl group having from one to the specified number of carbon atoms. Typically, C₁-C₆ alkyl groups include, but are not limited to, methyl, ethyl, n-propyl, iso-propyl, butyl, iso-butyl, sec-butyl, tert-butyl, pentyl, hexyl, and the like.

The term “C₂-Cₙ alkenyl” wherein n is an integer, as used herein, represents an olefinically unsaturated branched or linear group having from two to the specified number of carbon atoms and at least one double bond. Examples of such groups include, but are not limited to, 1-propenyl, 2-propenyl, 1,3-butadienyl, 1-butenyl, hexenyl, pentenyl, and the like.

The term “C₂-Cₙ alkynyl” wherein n is an integer refers to an unsaturated branched or linear group having from two to the specified number of carbon atoms and at least one triple bond. Examples of such groups include, but are not limited to, 1-propynyl, 2-propynyl, 1-butynyl, 2-butynyl, 1-pentynyl, and the like.

The term “C₃-Cₙ cycloalkyl” wherein n=8, represents cyclopropyl, cyclobutyl, cyclopentyl, cyclohexyl, cycloheptyl, and cyclooctyl.

As used herein the term “aryl” refers to an optionally substituted mono- or bicyclic carbocyclic ring system having one or two aromatic rings including, but not limited to, phenyl, benzyl, naphthyl, tetrahydronaphthyl, indanyl, indenyl, and the like. Optionally substituted aryl includes aryl compounds having from zero to four substituents, and A substituted aryl includes aryl compounds having one or more substituents. The term (C₅-C₈ alkyl)aryl refers to any aryl group which is attached to the parent moiety via the alkyl group.

The term “bicyclic” represents either an unsaturated or saturated stable 7- to 12-membered bridged or fused bicyclic carbon ring. The bicyclic ring may be attached at any carbon atom which affords a stable structure. The term includes, but is not limited to, naphthyl, dicyclohexyl, dicyclohexenyl, and the like.

The term “heterocyclic group” refers to an optionally substituted mono- or bicyclic carbocyclic ring system containing from one to three heteroatoms wherein the heteroatoms are selected from the group consisting of oxygen, sulfur, and nitrogen.

As used herein the term “heteroaryl” refers to an optionally substituted mono- or bicyclic carbocyclic ring system having one or two aromatic rings containing from one to three heteroatoms and includes, but is not limited to, furyl, thienyl, pyridyl and the like.

As used herein, the term “optionally substituted” refers to from zero to four substituents, wherein the substituents are each independently selected. Each of the independently selected substituents may be the same or different than other substituents.

The compounds of the present invention contain one or more asymmetric centers in the molecule. In accordance with the present invention a structure that does not designate the stereochemistry is to be understood as embracing all the various optical isomers, as well as racemic mixtures thereof.

The compounds of the present invention may exist in tautomeric forms and the invention includes both mixtures and separate individual tautomers. For example the following structure:

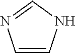

is understood to represent a mixture of the structures:

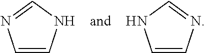 [SMILES: c1c[nH]cn1 ; "and" ; c1c[nH]cn1]

The terminology used herein is for the purpose of describing the particular versions or embodiments only, and is not intended to limit the scope of the present invention. All publications mentioned herein are incorporated by reference in their entirety.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Example 1

### Administration of Mitochondria after Cerebral Ischemia is Neuroprotectant

This Example relates to the evaluation of mitochondria therapy as a treatment modality for post-stroke recovery.

Animals models of stroke were prepared. Using the animal models at 10 days post-stroke, mice were treated with FUS to open the blood barrier followed by intra-arterial delivery of mitochondria. Thirty and sixty days after treatment, functional recovery of animals treated with FUS/mitochondria was assessed versus those treated with FUS alone, using standard behavioral and functional assays. The animals were sacrificed at the completion of experiments, the brain dissected and subjected to immunohistochemical analysis. This Example demonstrates the efficacy of mitochondria therapy as a modality for post-stroke recovery, as well as the efficacy of transplanted mitochondria to integrate into the brain at late time points after stroke.

### Methods Employed in Example 1

Mitochondria harvest. Mitochondrial isolation was performed as described by Preble et al., J Vis Exp. 2014; (91): 51682. Briefly, a skeletal muscle source was identified for mitochondrial harvest. Immediately prior to isolation, 4 mg of Subitilisin A and 20 mg of BSA were dissolved in 1 ml of Homogenizing Buffer. The isolation starts with a gastrocnemius muscle identification, followed by an anatomic harvest of all muscle with 2 cm of length and then, the fresh tissue was stored in 1×PBS. So, the harvest piece of muscle was transferred to a dissociation C tube containing 5 ml of cold Homogenizing Buffer (300 mM sucrose, 10 mM HEPES, and 1 mM EGTA, pH 7.4). The tissue was homogenized using the gentleMACS Octo Dissociator according to the manufacturer's instructions (Miltenyi Biotec, Auburn, Calif., United States of America). Next to dissociate the tissue, the C tube was placed on ice, and 250 μl of Subtilisin A (Sigma-Aldrich (St. Louis, Mo., United States of America); 9014-01-1) Stock Solution was added to the homogenate; it was mixed and incubated on ice for 10 minutes. When the time was done, a 40 μm cell strainer (Corning; 352340, Corning, N.Y., United States of America) was placed onto a 50 ml conic tube on ice and was used to filter the homogenate. Then, 250 μl of BSA Stock solution was added to the filtrate, mixed and the solution was passed through the other 40 μm cell strainer, placed onto the other 50 ml conic tube on ice. Next, a 10 μm cell strainer (PluriSelect; 43-50010-03, El Cajon, Calif., United States of America) was placed onto the 50 ml conic tube on ice and was used to filter the solution. Then, the filtrate was transferred to three pre-chilled 1.5 ml microfuge tube and centrifuged at 9,000×g for 10 minutes at 4° C. Finally, the supernatant was removed and re-suspended with 1 ml of cold Respiration Buffer.

Mitochondrial viability. ATP assay was used to assess functional viability of mitochondria and was summarized as described in CellTiter-Glo Luminescent Cell Viability Assay Protocol. Prior to use, the CellTiter-Glo Luminescent Cell Viability (Promega; G7570, Madison, Wis., United States of America) solution was thawed and equilibrated to room temperature. A Microplate, PS, 96 well, F-Botton, Lumitrack (Greiner, 655075, Monroe, N.C., United States of America) was prepared with triplicated sample of mitochondria, standards and medium without cells (to obtain a value for background luminescence). Next, the plate was allowed to equilibrate at room temperature for 10 minutes and then, a volume of CellTiter-Glo Reagent was added, equal to the volume of cell culture medium present in each well. The plate was mixed for 2 minutes on an orbital shaker to induce cell lysis, and then incubated at room temperature for 10 minutes to stabilize luminescent signal. Next, the plate was placed into the SpectraMax i3x Multi-Mode Microplate Readers (Molecular Devices, San Jose, Calif., United States of America) for 5 minutes and then, the cell viability was measured. ATP levels were expressed in nmol/g tissue.

Mitotracker staining. Prior to removing the supernatant from the microfuge the pellets of mitochondria were resuspended on the bottom with a 94 μl of Mitotracker Red CMXRos (Invitrogen; M7512, Carlsbad, Calif., United States of America) at a final concentration of 200 nM freshly prepared from stock solution 1 mM in DMSO. The solution was incubated at room temperature for 20 minutes. Next, mitochondria solution was divided in three microfuges 1.5 ml tube and washed with 1.5 ml of Respiration Buffer in each tube. Next, the tubes were centrifuged at 9,000×g for 10 minutes at 4° C. Finally, the pellets of labeled mitochondria were resuspended with 1 ml of Respiration Buffer.

Transient Proximal Middle Cerebral Artery Occlusion. The transient middle cerebral artery occlusion (tMCAO) was performed as described by Kozuimi et al., Jpn J Stroke. 1986; 8:108. Briefly, all main arteries involved in blood supply of the brain were exposed, including the common carotid artery, external carotid artery, and internal carotid artery. The external carotid artery was knotted with 6-0 silk sutures, followed by common carotid artery and then, a microclip, 10 g pressure (WPI; 15911, Sarasota, Fla., United States of America) was positioned around the internal carotid artery. Next, a small incision was done in common carotid artery using a micro-scissor and then, a 6.0 monofilament (Doccol Corp; 6022910PK10, Sharon, Mass., United States of America) was inserted into the internal carotid artery. The filament was advanced until a resistance was felt, demonstrating that the middle cerebral artery was occluded. At 1 hour after onset of middle cerebral artery occlusion, animals were re-perfused, and the common carotid artery kept permanently knotted.

Distal Permanent Middle Cerebral Artery Occlusion. The distal permanent middle cerebral artery occlusion model was performed as described by Doyle et al., Methods Mol Biol. 2014; 1135:103-10. Mice were anaesthetized in 2-3% isoflurane anesthesia induction box. Then, he side of the mouse's head was shaved between the ear and the eye, on the side wished to be occluded. Next the mouse was placed on prone positing on top of a feedback-controlled heating blanket to maintain mouse temperature at 37° C. and a rectal thermometer was inserted to control the temperature of the heating blanket. The skin was prepped for surgery by swabbing with a solution of chlorhexidine, and then rinsing the area with sterile saline.

Next, scissors were used to make a 4 mm horizontal incision in the skin between the orbit and the auditory canal. This exposed the coronal suture and the temporalis muscle. The temporalis muscle was incised horizontally in whole its superior edge and, vertical on the superior part of the posterior limit using scissors. Then, the temporalis muscle was tied with Vycril™ suture (Ethicon, Bridgewater, N.J., United States of America) and retracted to anterior position to expose the skull. Next, a hand-held drill was used to create a 2×3 mm diameter rectangle craniotomy directly over the MCA. So, the microforceps (0.05×0.01 mm diameter) were used to remove the meninges and then cauterize the MCA with a bipolar forceps (Malis®; 08-0099, Symmetrical Surgery Inc., Antioch, Tenn., United States of America) attached to an electrosurgical generator (Valleylab force 2, available from Medtronic Inc., Minneapolis, Minn., United States of America). Finally, once the MCA has been cauterized, the brain surface was rinsed with saline and the temporalis muscle and skin were folded back into place. Thus, the skin was sutured, and the mice injected with buprenorphine (0.1 mg/kg, subcutaneously) to provide analgesia.

Intra-Arterial Delivery of Mitochondria. Isolated mitochondria were intra-arterially delivered after removal of the filament placed in the middle cerebral artery by placing a micro-catheter into the internal carotid artery using a small arteriotomy in the common carotid artery in the neck.

Stereotactic Delivery of Mitochondria. Stereotactic delivery of isolated mitochondria was performed as described by Cetin et al., Nat Protoc. 2007; 1:3166-3173. Briefly, mice were anaesthetized by Isoflurane 2% and the head was shaved and the animal placed in the stereotaxic apparatus. Then, the ear bars were positioned in order to lead its ear canal onto the ear bar and the fixation of the system was performed. Small forceps were used to pull down the animal's lower jaw, slowly move the incisor adapter into the animal's mouth until the animal's incisors ‘fit’ in the opening of the adapter, then gently pulled back slightly and fixed the adaptor in place. A dissecting microscope at a low magnification (×10 to ×20) was used to visualize the top of the animal skull. The Asepsis procedure was performed prior to make a midline incision with small surgical scissors or scalpel. The subcutaneous and muscle tissue were separated and the bregma and lambda areas were gently cleaned. The head of the animal was leveled by measuring the z coordinates of bregma and lambda and adjusting the head position so that they become equal. The position of the x and y coordinates of bregma were measured and the coordinates of the target injection area were calculated (subtracted), as determined from a stereotaxic brain atlas. So, a small burr hole was performed over the target area using a hand-held drill. The 5 μl Hamilton syringe with mitochondria solution was attached to the stereotaxic apparatus. The micropipette was brought to the correct x and y position and lowered until it touches the exposed dura. After penetrating the dura, the micropipette was slowly lowered to the desired z coordinate of the injection site and then, began to slowly apply pressure with the syringe to inject the mitochondria solution. The speed and volume of the injection was controlled. Waited 2-3 min before withdrawing the needle; then withdrew slowly to avoid backflow of the mitochondria solution. The skin was sutured and triple antibiotic ointment was applied to the wound. The anesthetic lidocaine was injected subcutaneously near the wound for local anesthesia during the early recovery period (Cetin et al., Nat Protoc. 2007; 1:3166-3173).

Bubble Preparation.

Cationic Lipid-Shelled Microbubble (MB) Fabrication. To synthesize the cationic lipid-shelled MBs, a mixure of 2 mg/ml 1,2-distearoyl-sn-glycero-3-phosphocholine (DSPC; Avanti Polar Lipids, Alabastar, Ala., United States of America), 2 mg/ml polyethylene glycol 6000 monostearate (PEG 6000 MS; Stepan Kessco, Northfield, Ill., United States of America), and 0.8 mg/ml 1,2-distearoyl-3-trimethylammonium-propane (DSTAP; Avanti Polar Lipids, Alabastar, Ala., United States of America) was sonicated in 0.9% NaCl (Baxter, Deerfield, Ill., United States of America) to create a micellar emulsion. The mixture was then filtered through a 0.2 um Nylon sterile filter. One ml of this lipid mixture was added to a 2 ml 13 mm glass vial, and the headspace of the vial was filled with decafluorobutane gas (F2 Chemicals Ltd; Preston, United Kingdom), and then the vial was sealed and sonicated at high power (20 kHz, 30 s) with an ultrasound disintegrator (XL2020; Misonix, Farmingdale, N.Y., United States of America) to generate the microbubbles.

The MBs were cleaned by flotation centrifugation before each experiment to remove residual micelles. An aliquot of the MB solution was centrifuged at 1000 rpm for 10 minutes, and the infranatant was removed and the bubbles resuspended in degassed saline. This process was repeated three times before the final resuspension of the bubbles at a concentration between 1.5 and 2*10⁹ bubbles/ml. MBs were sized and counted using a Coulter counter (Multisizer 3; Beckman Coulter, Fullerton, Calif., United States of America).

High-Frequency Focused Ultrasound. High-intensity focused ultrasound (FUS) was performed using a modification as reported by Bing et al., Int J Hyperthermia. 2015; 31(8):813-22.

Benchtop Focused Ultrasound.

Stereotactic FUS-Mediated Mitochondria Delivery. Sonications using the stereotactic frame were performed using a 1 MHz spherical-face single element FUS transducer with a diameter of 4.5 cm (Olympus; Center Valley, New Jersey, United States of America). FUS (0.3 MPa; 120 seconds, 10 ms bursts, 0.5 Hz burst rate) was targeted to the right striatum. The 6-dB acoustic beamwidth along the axial and transverse directions are 15 mm and 4 mm, respectively. The waveform pulsing was driven by a waveform generator (AFG310; Tektronix, Bracknell, United Kingdom) and amplified using a 55 dB RF power amplifier (ENI 3100LA; Electronic Navigation Industries, Richardson, Tex., United States of America).

Mice were anesthetized with an intraperitoneal injection of 120 mg/kg ketamine, 12 mg/kg xylazine, and 0.08 mg/kg atropine in sterilized 0.9% saline. A catheter was previously inserted into the ICA to permit intravenous injections of MBs and mitochondria. The heads of the mice were shaved and depilated, and the animals were then positioned prone in a stereotactic frame (Stoelting, Wood Dale, Ill., United States of America). The mouse heads were ultrasonically coupled to the FUS transducer with ultrasound gel and degassed water, and positioned such that the ultrasound focus was localized to the right striatum. Mice received an intra-arterial injection of the cleaned MBs (2×10⁵ MBs/g body weight), followed by injection of 0.1 mL of 2% heparinized saline to clear the catheter. Sonication began immediately after clearance of the catheter. Mitochondria were delivered intra-arterial after completion of the sonication sequence. Animals were then removed from the stereotactic frame and placed on a warm pad for 30 minutes prior to reversal of the anesthetic with antisedan (1 mg/ml).

2,3,5-Triphenyltetrazolium chloride (TTC) Staining. TTC staining was performed as described by Benedek et al., Brain Res. 2006 Oct. 20; 1116(1):159-65. In short, mice were euthanized after 24 hours of distal coagulation of Middle Cerebral Artery with an intraperitoneal (i.p.) injection of Euthasol and then, heads were removed and skulls were quickly stripped. The brains were harvested and sliced in coronal sections with 1 mm, using the brain matrix. Next, the slices were placed into the TTC solution (500 mg of TTC mixed with 25 ml of PBS 1×) and kept for 25 minutes. Finally, the slices were moved to a 4% paraformaldehyde. The size of the stroke was measured using the plot profile function of FIJI (United States National Institutes of Health (NIH), Bethesda, Md., United States of America).

H&E staining. Mice were euthanized with an i.p. injection of Euthasol and perfused with 20 ml of PBS 1× and next, perfused with 20 ml of 4% paraformaldehyde for fixation. Skin was removed from the head and the muscle were stripped of the bone. Then, the top of the skull was removed with surgical scissors and the brain harvested. Next, the brain was placed in a 4% paraformaldehyde solution for four hours and then changed to sucrose gradient dehydration, followed by freezing in vinyl molds embedded in O.C.T. compound at −80° C. until the cryosection. So, the 10 μm-thick coronal sections were sliced using a cryostat (Leica, Buffalo Grove, Ill., United States of America) and mount on microscope slides (Fisherbrand; 12-550-15, Pittsburgh, Pa., United States of America) and kept freeze in −20° C. The slides were placed in a slide holder and then stained with filtered 0.1% Mayers Hematoxylin (Sigma; MHS-16, St. Louis, Mo., United States of America) for 1 minute. In a Coplin jar, the slides were rinsed in cool running ddH2O for 5 minutes and next dipped in 0.5% Eosin (1.5 g dissolved in 300 ml of 95% EtOH) 12 times, followed by a dip in distilled H₂O until the eosin stops streaking. Next, the sections were dipped in 50% EtOH (10 times) and, next in 70% EtOH (10 times). After, the slides were equilibrated in 95% EtOH for 30 seconds and, next in 100% EtOH for 1 minute. Finally, the slides were dipped in Xylene several times and then mounted and coverslipped with DPX Mounting Medium (Electron Microscopy Sciences; 13512, Hatfield, Pa., United States of America).

Immunohistochemistry. Immunohistochemistry was performed with nuclear (DAPI), neuronal (MAP2, NeuN), glial (GFAP) and microglial (Iba-1) markers as described by Ramos-Vara, Methods Mol Biol. 2011; 691:83-96. Mice were euthanized with an i.p. injection of Euthasol and perfused with 20 ml of PBS 1× and next, perfused with 20 ml of 4% paraformaldehyde for fixation. Skin was removed from the head and the muscle were stripped of the bone. Then, the top of the skull was removed with surgical scissors and the brain harvested. Next, the brain was placed in a 4% paraformaldehyde solution for four hours and then changed to sucrose gradient dehydration, followed by freezing in vinyl molds embedded in O.C.T. compound at −80° C. until the cryosection. So, the 20 μm-thick coronal sections were sliced using a cryostat (Leica, Buffalo Grove, Ill. United States of America) and mounted on microscope slides (Fisherbrand; 12-550-15, Pittsburgh, Pa., United States of America) and kept frozen in −20° C.

Whole mounts were incubated with PBS containing 10% of normal serum (either goat or donkey), 0.2% Triton-X-100 for 2h at room temperature (RT), followed by incubation with PBS containing 2% of normal serum (either goat or donkey), 0.2% Triton-X-100 and appropriate dilutions of primary antibodies: CD31 (DSHB; clone P2B1; 1:50, Iowa City, Iowa, United States of America), GFAP (Invitrogen; Polyclonal; 1:1000, Carlsbad, Calif., United States of America), MAP2 (Sigma; clone HM-2; 1:500, St. Louis, Mo., United States of America), Iba-1 (abcam; Polyclonal; 1:500, Cambridge, Mass., United States of America) 0/N at 4° C. Whole mounts were then washed 3 times for 5 min at RT in PBS followed by incubation with Alexa-fluor 488/594/647 chicken/goat anti rabbit/goat/mouse IgG antibodies (Invitrogen, 1:500, Carlsbad, Calif., United States of America) for 2h at RT in PBS with 2% BSA and 0,2% Triton-X-100. After 2 hours, the microscope slides were mount with ProLong Gold antifade reagent with DAPI (Invitrogen; P36935, Carlsbad, Calif., United States of America) under coverslips.

ATP Assay. The ATP assay was used to assess functional viability of mitochondria and is summarized as described in Preble et al., J Vis Exp. 2014; (91): 51682. The kit was equilibrated to room temperature. A 10 mM ATP Stock Solution was prepared by dissolving lyophilized ATP pellet in 1,170 μl of double distilled water. The sample was stored on ice. Next, 5 ml of Substrate Buffer solution was added to a vial of lyophilized substrate solution. 100 μl of Respiration Buffer was added to wells of a black, opaque bottom, 96 well plate or other plate as system permits. Next, 10 μl of isolated mitochondria was added to each well. 50 μl of mammalian cell lysis solution was added to wells. Standards and controls should be used. The plate was incubated at 37° C. for 5 min on a shaker at 125 rpm. ATP standards in concentrations of 0.1 mM, 0.05 mM, 0.01 mM, 0.005 mM, 0.001 mM, and 0.0001 mM ATP were prepared from the 10 mM ATP Stock Solution and stored on ice. 10 μl of ATP standards were added to corresponding wells after appropriate period of incubation. 50 μl of the reconstituted substrate solution was added to each well and incubated at 37° C. on shaker for 5 min at 125 rpm. The samples were promptly read on a spectrophotometer.

For the determination of the ATP levels (Cristobal, J. D. et al., Journal of Neurochemisty 79, 4156-459 (2008), mice were sacrificed 12 h after MCAO surgery plus vehicle or mitochondria injection (intra-arterial or stereotactic or intra-arterial combined with FUS) and immediately immersed into liquid nitrogen. Also, healthy mice without intervention were sacrificed as described before. Once frozen, brains were quickly taken out and stored at −80° C. until ATP determination. To avoid postmortem degradation, the skulls were quickly stripped on aluminum foil placed on the dry ice mixed with absolute ethanol and the brain samples were dissected and homogenized in a medium containing 0.3% (w/v) trichloroacetic acid and 1 mM EDTA. The homogenate was centrifuged at 10,000 g for 3 min at 4° C. and the supernatant was mixed 1:1 with Tris-acetate buffer solution (7.75). Brain ATP levels were determined using a CellTiter-Glo Luminescent Cell Viability, and was measured in the SpectraMax i3x Multi-Mode Microplate Readers (Molecular Devices, San Jose, Calif., United States of America). ATP levels were expressed in nmol/g tissue.

MRI. Magnetic resonance imaging protocols to assess volume of infarction, opening of blood brain barrier and hemorrhage were performed as described by Denic et al., Neurotherapeutics. 2011 January; 8(1): 3-18.

MR-Guided FUS-Mediated Mitochondria Delivery. Mice were anesthetized with an intraperitoneal injection of 120 mg/kg ketamine, 12 mg/kg xylazine, and 0.08 mg/kg atropine in sterilized 0.9% saline. A catheter was previously inserted into the ICA to permit intra-arterial injections of MBs and mitochondria. The heads of the mice were shaved and depilated, and the animals were then placed in a supine position over a degassed water bath coupled to an MR-compatible small animal FUS system (RK-100; FUS Instruments, Toronto, Canada). The entire system was then placed in a 3T MR scanner (Magnetom Trio; Siemens Medical Solutions, Malvern, Pa., United States of America). A 2 inch cylindrical transmit-receive RF coil, designed and built in-house, was placed around the mouse's head to maximize imaging SNR. Baseline T1-weighted images were acquired and used to select 4 FUS target locations in and around the right striatum.

Mice received an injection of the MBs (2×10⁵ MBs/g body weight), followed by 0.1 mL of 2% heparinized saline to clear the catheter. Sonication began immediately after clearance of the catheter. Sonications were performed at 0.3 MPa using a 1.1 MHz single element focused transducer (FUS Instruments, Toronto, Canada) operating in 10 ms bursts, 0.5 Hz pulse repetition frequency and 2 minutes total duration. Immediately following the FUS treatment, mice received an intra-arterial injection of the mitochondria, then Gd-DPTA contrast agent (0.5 ul/g body weight; Magnevist; Bayer Health Care, Indianola, Pa., United States of America), and T1-weighted contrast-enhanced images were acquired to assess BBB opening. Animals were removed from the MRI and placed on a warm pad for 30 minutes prior to reversal of the anesthetic with antisedan (1 mg/ml)

Flow Cytometry. Flow cytometry with neuronal, astrocytic and mitochondrial markers were performed using the methods described by Martin et al., ACS Chem Neurosci. 2017 Feb. 15; 8(2):356-367.

Mice were euthanized with an i.p. injection of Euthasol and perfused with 20 ml of PBS 1×. Next, skin was removed from the head and the muscle were quickly stripped of the bone. Then, the top of the skull was quickly removed with surgical scissors and the brain harvested. Then, the brains were placed in conic tubes with 5 ml of Neurobasal medium+B27 (GlutaMAX Supplement to 0.5 mM concentration and 2% B-27) on ice. The stroke hemisphere was harvested in Neurobasal medium and then weigh. After that, the stroked hemisphere was transferred into the C tube containing 2 ml of papain digestion buffer (Total Volume=5 ml-11 μl of 0.5 M EDTA, 50 μl of B-mercaptoethanol 100x solution, 50 μl of L-cysteine-HCl solution (3.14 mg of L-cysteine-HCl diluted in 50 μl, Earle's Balanced Salt Solution) and 4.889 ml of Neurobasal A medium). Next, the C Tube was placed onto the gentleMACS Dissociator and run program “m_brain_01” and then added 4 U of Papain/ml of brain solution. The samples were incubated at 37° C. for 15 minutes with rotation and then placed the C Tube onto the gentleMACS Dissociator and run program “m_brain_02”. Next, the samples were incubated at 37° C. for 10 minutes with rotation and then placed the C Tube onto the gentleMACS Dissociator and run program “m_brain_03”. After, the samples were incubated at 37° C. for 10 minutes with rotation. When it is done, cells were passed through 70 μm nylon filter and washed with 1 mL of NB-B27 medium and next spun cells at 300×g for 5 minutes at 4° C. the supernatant was aspirated and cells re-suspended with 5 ml of fresh Isotonic Percoll 30%. After, the Percoll gradient was spun at 400×g for 20 minutes at room temperature without acceleration and deceleration. Finally, the supernatant was aspirated and cells re-suspended into appropriate volume of FACS buffer (pH 7,4; 0.1M PBS; 1 mM EDTA: 1% BSA).

An aliquot of unstained cells of each sample was counted using Cellometer Auto2000 (Nexcelor, Lawrence, Mass., United States of America) to provide accurate counts for each population.

Cells were stained for extracellular marker with antibodies to Live/Dead-Acqua (Invitrogen, Carlsbad, Calif., United States of America), CD45-Alexa-647 (Biolegend, San Diego, Calif., United States of America) and CD11b-BV421 (Biolegend, San Diego, Calif., United States of America). Then, the cells were fixed and permeabilized using the eBioscience Foxp3/Transcriptn kit (Invitrogen; 00-5523-00, Carlsbad, Calif., United States of America). Next, cells were stained for intracellular marker with antibody to Recombinant Anti-NeuN-FITC (abcam, Cambridge, Mass., United States of America).

Fluorescence data were collected with a Gallios (Beckman Coulter, Indianapolis, Ind., United States of America) then analyzed using Flowjo software (Treestar, Ashland Oreg., United States of America). Data processing was done with Excel and statistical analysis performed using Prism 7.0a (GraphPad Software, Inc., San Diego, Calif., United States of America).

Animals (DSRed and Black 6). Male or female wild-type mice (C57BL/6J background) were either bred in-house, purchased from the Jackson Laboratory (Bar Harbor, Me., United States of America). Only adult animals (eight to ten weeks) were used in this study and animals from different cages in the same experimental group were selected to assure randomization.

Also, a transgenic mouse with a MitoTimer report was used in this study. Laker et al. (Laker, R. C. et al., Journal of Biological Chemistry 289, 12005-12015 (2014)) engineered a pMitoTimer reporter gene by targeting a fluorescent Timer protein to mitochondria by adding the mitochondrial targeting sequence of the cytochrome c oxidase subunit VIII gene to the N terminus of the coding region of Timer, under control of the constitutive CMV promoter. Timer encodes a DsRed mutant (DsRed1-E5) that fluoresces like green fluorescence protein when newly synthesized, and shifts the fluorescent spectrum irreversibly to red following a form of oxidation (dehydrogenization) of the Tyr-67 residue.

Mice of all strains were housed in identical housing conditions where an environment has controlled temperature and humidity, on 12 hours light/dark cycles (lights on at 7:00), and fed with regular rodent's chow and sterilized tap water. All experiments were approved by the Institutional Animal Care and Use Committee of the University of Virginia, Charlottesville, Va., United States of America.

Confocal Imaging. Images were acquired with a Leica TCS SP8 confocal system (Leica Microsystems, Buffalo Grove, Ill., United States of America) using the LAS AF Software. For the images of the complete brain coronal section, images were acquired using a 20x objective with 0.70 NA. For the images of cells confocal images were acquired with a 40x oil immersion objective with 1.30 NA or 63x oil immersion objective with 1.40 NA. All images were acquired with at a 1024×1024-pixel resolution. Quantitative assessments were performed using FIJI software (United States National Institutes of Health (NIH), Bethesda, Md., United States of America) and statistical analyses were performed using GraphPad Prism software.

Electron Microscopy. Mitochondria were isolated as previously described and fixed in 2.5% glutaraldehyde, 2% paraformaldehyde in 0.1M sodium cacodylate buffer, pH 7.4, and post-fixed in 2% osmium tetroxide in 0.1M cacodylate buffer with 0.15% potassium ferrocyanide. After rinsing in buffer, the tissue was dehydrated through a series of graded ethanol to propylene oxide, infiltrated and embedded in epoxy resin and polymerized at 70° C. O/N. Semi-thin sections (0.5 microns) were stained with toluidine blue for light microscope examination. Finally, it was imaged using the Tecnai F20 TEM with an UltraScan CCD camera (Advanced Microscopy core, University of Virginia, Charlottesville, Va., United States of America).

Evans Blue (EB) injection and quantification. Evans blue (Sigma-Aldrich, St. Louis, Mo., United States of America) was injected through the internal carotid artery into the mice. After 4 hours, the mice were euthanized with an intraperitoneal (i.p.) injection of Euthasol and perfused with 20 ml of PBS 1×. Then, the brain was harvested and sliced in coronal sections with 1 mm, using the brain matrix. Finally, the slices were scanned and then, the intensity of the Evans Blue was measured using the plot profile function of FIJI (United States National Institutes of Health (NIH), Bethesda, Md., United States of America).

Results and Discussion

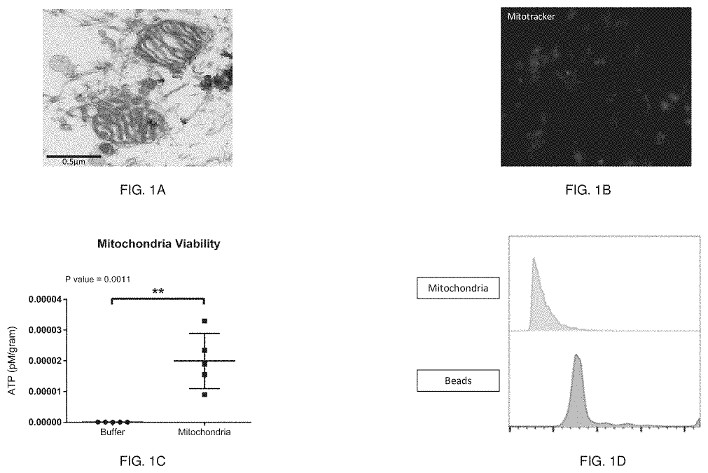

Mitochondrial isolation, staining and activity was assessed. 10⁹ biochemically active mitochondria are harvested from two punch biopsies (FIGS. 1A-1D). These samples were functionally active (FIG. 1C), possessed the appropriate transmission electron microscopic features (FIG. 1A), and could be stained with fluorescent dyes for in vivo tracing (FIGS. 1B and 1D).

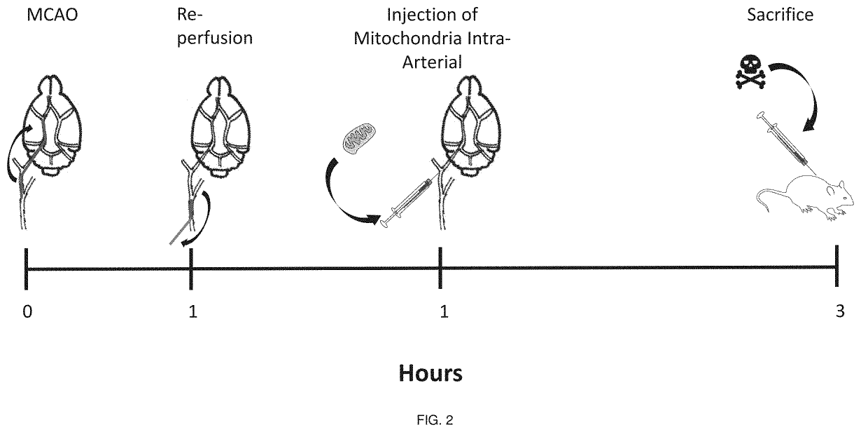

In each case in the preparation of a stroke model, n=6-9 C57 Black6 animals aged 8-12 weeks were used. The transient middle cerebral artery occlusion or the permanent distal middle cerebral artery occlusion model were used to create ischemic injury. The timing of ischemia and delivery of mitochondria (intra-arterial or stereotactic) is depicted in FIG. 2.

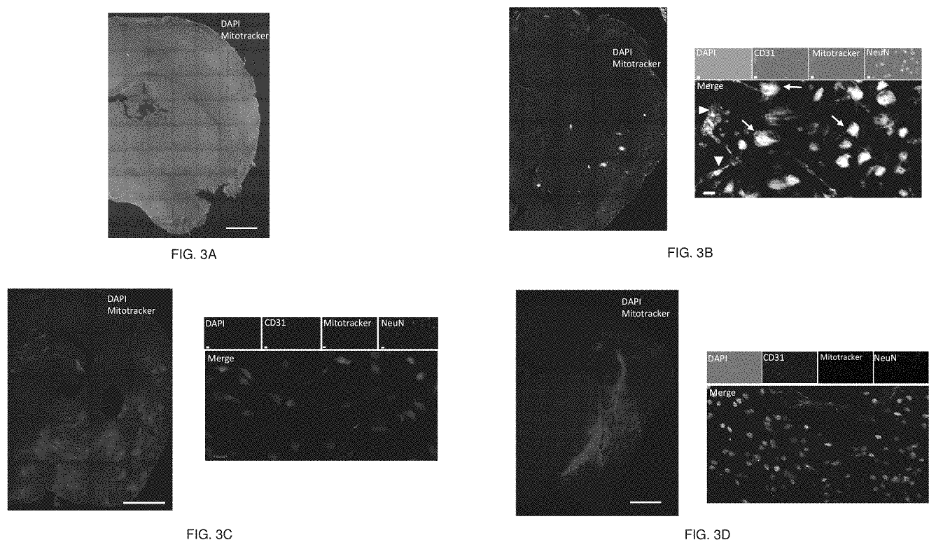

Mitochondria traverse the Blood Brain Barrier (BBB) to enter the brain, and this process is enhanced after stroke and further improved upon by focused ultrasound (FUS). As demonstrated in FIGS. 3A-3D, mitochondria were able to traverse the blood brain barrier after stroke and the process of mitochondrial delivery was enhanced by high-frequency focused ultrasound.

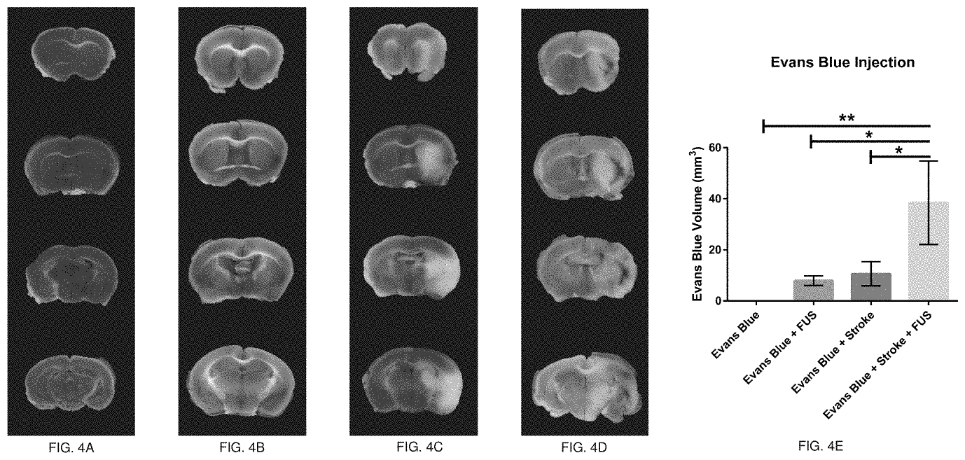

BBB opening after stroke is enhanced by FUS. To assess degree of BBB opening with FUS and stroke, TTC staining was performed and volume of BBB opening was assessed as depicted in FIGS. 4A-4E.

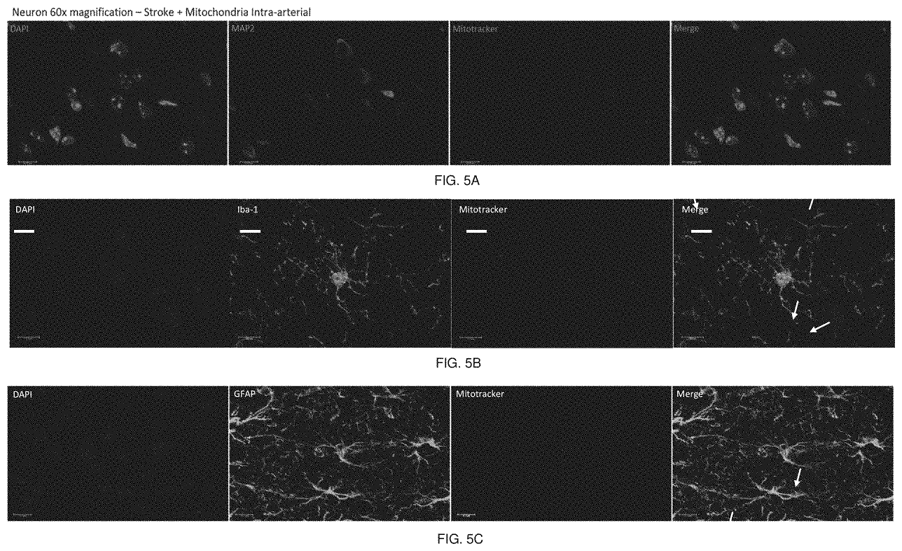

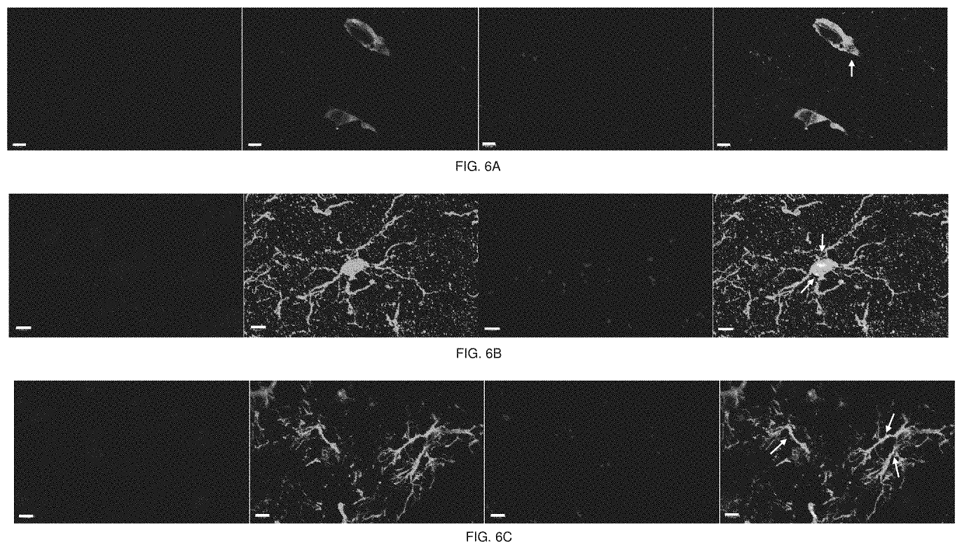

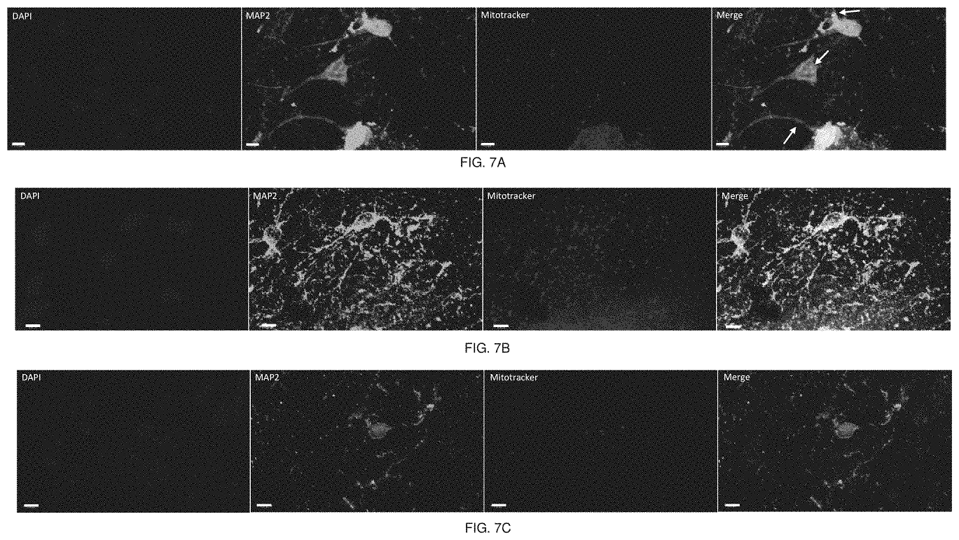

The central nervous system's cells pickup mitochondria upon delivery. As depicted in FIGS. 5A-7C, cells of the central nervous system engulf the mitochondria regardless of method of delivery (intra-arterial or stereotactic).

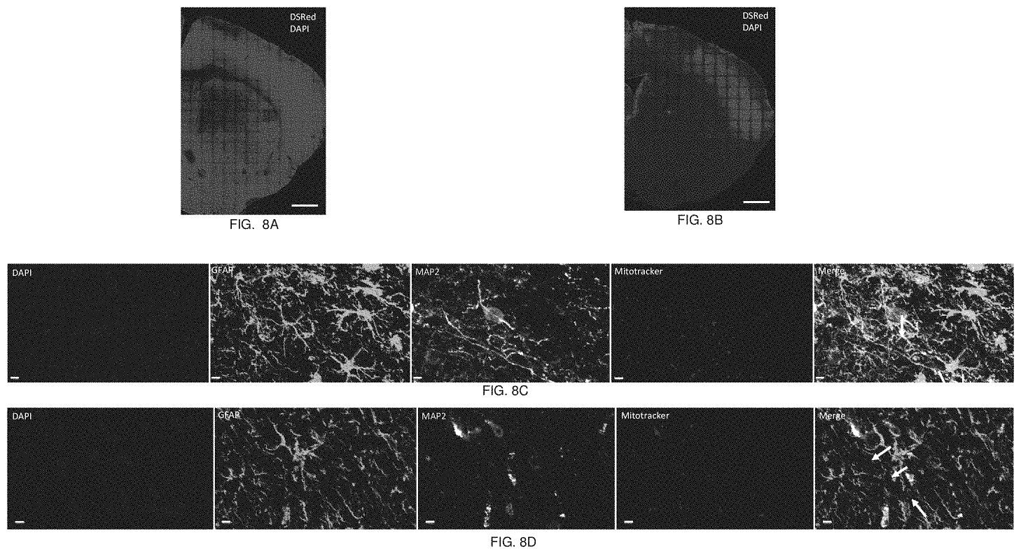

Results of mitochondria delivery were confirmed using genetically-labelled mitochondria. To assess the reliability of the present results, the results of transplantation of mitochondria were confirmed with an alternative model whereby mitochondria are genetically labelled with DS-Red (FIGS. 8A-8D).

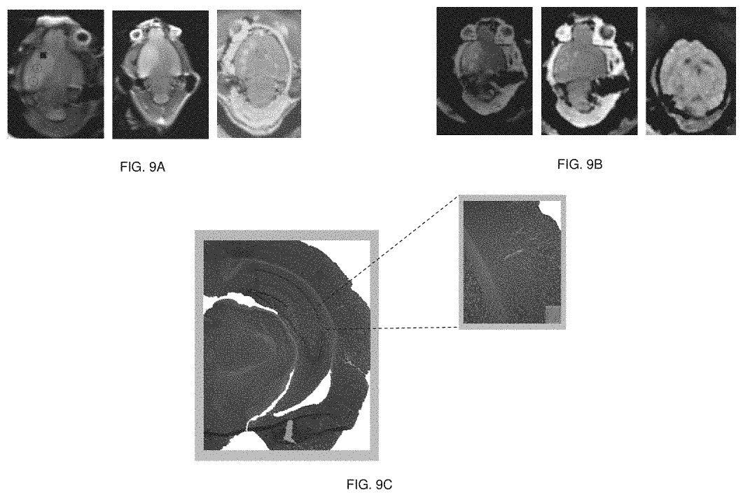

High-frequency focused ultrasound (FUS) does not result in haemorrhage after stroke. To assess if high-frequency focused ultrasound (FUS) could be safely applied to an ischemic stroke bed, evidence of haemorrhage was assessed on both MRI and immunohistochemistry (FIGS. 9A-9C). The standard settings used in these experiments do not lead to haemorrhage in the stroke bed.

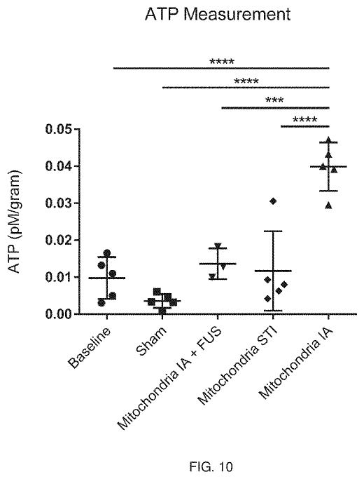

Transplantation of mitochondria increases the concentration of ATP in the stroked hemisphere. To assess if mitochondrial delivery increased ATP load in the stroked hemisphere, an ATP assay was performed in the stroked hemispheres after mitochondria delivery (intra-arterial or stereotactic). Transplantation of mitochondria indeed increased concentration of ATP in the targeted hemisphere (FIG. 10).

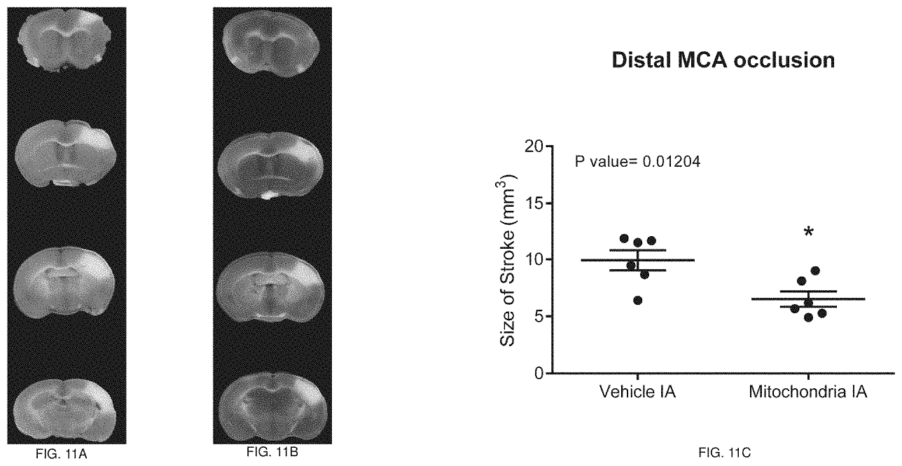

The ischemic core is reduced following mitochondria transplantation. To assess the effect of mitochondrial delivery on size of stroke, TTC staining was performed and a significant shrinkage of the ischemic core following transplantation of mitochondria was observed (FIGS. 11A-11C). Thus, unexpected results are shown in FIGS. 11A-11C using a model of temporary middle cerebral artery occlusion in mice where it is demonstrated that administration of intra-carotid mitochondria after opening of the blood brain barrier using focused ultrasound resulted in a decrease in the volume of infarcted brain.

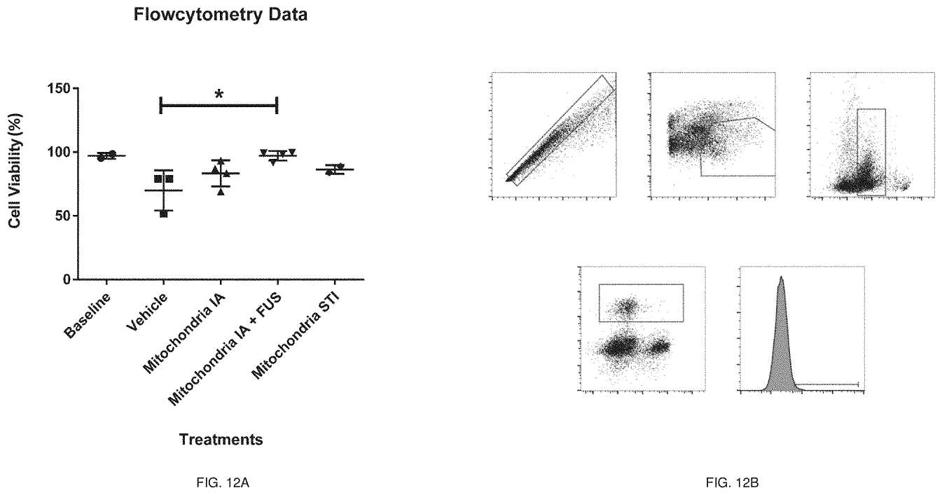

Cellular survival is enhanced in the hemispheres that receive mitochondria. To assess cellular survival, flow cytometry was performed on hemispheres that received mitochondria and controls (FIGS. 12A and 12B). Cells that engulfed mitochondria were more likely to survive ischemic injury.

### Example 2

### Delivery of Precursor Cells

This Example utilizes focused ultrasound (FUS) to selectively open the blood brain barrier (BBB) at the site of stroke-induced injury. A prospectively isolated and purified population of mouse neural stem cells (NSC) tagged with a fluorescent marker are systemically delivered (e.g., by an intra-arterial approach) and the rate of integration of this population of cells into the site of selective BBB opening and in the rest of the brain is studied. A control group is used to assess the efficacy of FUS-induced BBB opening. Using FUS to safely and selectively open the BBB for systemically administered cell transplantation into the CNS provides a minimally invasive modality for treatment and regeneration in the post-stroke period.

Cerebral ischemia is generated in mice by performing selective middle cerebral artery occlusion (MCAO) as described in Example 1. A control group is used. Magnetic resonance imaging (MRI) is used to document the volume of infarct in these animals on post-stroke day 3. At 10 days post-stroke, FUS is used to open the BBB at the site of stroke-induced injury while delivering 10⁹ prospectively isolated, fluorescently tagged, fetal mouse neural stem cells (or controls) intravenously. At 20 days post-stroke, the animals are sacrificed, the brain dissected and subjected to immunohistochemical analysis in accordance with techniques described in Example 1. The degree of integration of the fluorescently tagged cells into the site of BBB opening and in the rest of the brain between the FUS and sham-treated groups is assessed. Successful completion shows the safety and efficacy of FUS in selectively opening the BBB in the post-stroke settings.

Using the animal models generated as described above, at 30 and 60 days post-stroke, functional recovery of animals treated with FUS/NSC is assessed versus those treated with FUS alone, NSC alone and those treated without FUS or NSC using standard behavioral and functional assays. The animals are sacrificed at the completion of experiments, the brain dissected and subjected to immunohistochemical analysis in accordance with techniques described in Example 1. Successful completion demonstrates the efficacy of combination FUS/NSC therapy as a modality for post-stroke recovery and the efficacy of transplanted NSCs to integrate and differentiate into the brain at late time points after stroke.

### Example 3

### Patient Treatment

When a patient arrives in the ER, a stroke is identified and the subject is taken to the angiography suite to revascularize them (this is all standard of care). At the same time, a punch biopsy of skeletal muscle is taken and mitochondria are processed from this tissue. This can be done in the angiography suite in 30 minutes with minimal additional equipment necessary. Once the blood vessel is opened, using the existing catheter that has been placed into the artery for removal of clot, mitochondria are delivered.

### BIBLIOGRAPHY

1. Berkhemer O A et al. (2015) A randomized trial of intraarterial treatment for acute ischemic stroke. N Engl J Med 372:11-20.
2. Elias W J et al. (2016) A Randomized Trial of Focused Ultrasound Thalamotomy for Essential Tremor. N Engl J Med 275:730-9.
3. Kang S K et al. (2003) Improvement of neurological deficits by intracerebral transplantation of human adipose tissue-derived stromal cells after cerebral ischemia in rats. Exp Neurol 183:55-366.
4. Kim S U et al. (2009) Stem cell-based cell therapy in neurological diseases: A review. J Neurosci Res 87:2183-2200.
5. Steinberg G K et al. (2016) Clinical outcomes of transplanted modified bone marrow-derived mesenchymal stem cells in stroke: A phase 1/2a study. Stroke 47:1817-1824.
6. Tyrnianski M. (2013) Novel approaches to neuroprotection trials in acute ischemic stroke. Stroke 44(10):2942-50.
7. The National Institute of Neurological Disorders and Stroke rt-PA Stroke Study Group. (1995). Tissue Plasminogen Activator for Acute Ischemic Stroke. N Engl J Med 333:1581-1588.
8. VDH Division of Population Health Data, Virginia Health Information, 2017.
9. Preble et al., J Vis Exp. 2014; (91): 51682.
10. Benedek, A. et al. Use of TTC staining for the evaluation of tissue injury in the early phases of reperfusion after focal cerebral ischemia in rats. Brain Research 1116, 159-165 (2006).
11. Cristobal, J. D. et al. Neuroprotective effect of aspirin by inhibition of glutamate release after permanent focal cerebral ischaemia in rats. Journal of Neurochemistry 79, 456-459 (2008).
12. Cetin, A., Kornai, S., Eliava, M., Seeburg, P. H. & Osten, P. Stereotaxic gene delivery in the rodent brain. Nature Protocol 1, 3166-3173 (2006).
13. Laker, R. C. et a/. A NovelMitoTimerReporter Gene for Mitochondrial Content, Structure, Stress, and Damagein Vivo. Journal of Biblogical Chemistry 289, 12005-12015 (2014).

The disclosures of each and every patent, patent application, and publication cited herein are hereby incorporated by reference herein in their entirety.

Headings are included herein for reference and to aid in locating certain sections. These headings are not intended to limit the scope of the concepts described therein under, and these concepts may have applicability in other sections throughout the entire specification.

While this invention has been disclosed with reference to specific embodiments, it is apparent that other embodiments and variations of this invention may be devised by others skilled in the art without departing from the true spirit and scope of the invention.

What is claimed is:

1. A method of treating stroke in a subject, the method comprising administering to the subject an effective amount of a composition comprising mitochondria, precursor cells, or combinations thereof, in a manner in which the composition is delivered to a revascularized bed of tissue in the subject's brain.

2. The method of claim 1, wherein the revascularized bed of tissue is provided by opening an occluded blood vessel in the brain of the subject

3. The method of claim 1, wherein the opening of an occluded blood vessel is accomplished by thrombectomy or thrombolysis.

4. The method of claim 1, where the mitochondria and/or the precursor cells are autologous to the subject.

5. The method of claim 1, wherein the precursor cells are neural precursor cells, mesenchymal precursor cells, or a combination thereof.

6. The method of claim 1, wherein the administering of the composition comprises intra-arterial administration or stereotactic injection.

7. The method of claim 6, wherein the intra-arterial administration comprises administration to an internal carotid artery, a vertebral artery, and/or to a branch thereof.

8. The method of claim 1, wherein the composition further comprises a neuroprotective agent.

9. The method of claim 1, wherein the composition further comprises a pharmaceutically acceptable carrier.

10. The method of claim 9, wherein the pharmaceutically acceptable carrier is pharmaceutically acceptable for use in humans.

11. The method of claim 1, comprising opening the blood-brain barrier prior to, during, and/or after the administering of the composition.

12. The method of claim 11, wherein opening the blood-brain barrier comprises exposing the subject to focused ultrasound and/or to intra-arterial delivery of mannitol prior to, during, and/or after the administering of the composition.

13. A pharmaceutical composition for use in treating stroke, the composition comprising mitochondria, precursor cells, or a combination thereof.

14. The pharmaceutical composition of claim 13, wherein the pharmaceutical composition is adapted for delivery to a revascularized bed of tissue in the subject's brain.

15. The pharmaceutical composition of claim 13, wherein the mitochondria and/or the precursor cells are autologous to a subject to whom the pharmaceutical composition will be administered.

16. The pharmaceutical composition of claim 13, wherein the precursor cells are neural precursor cells, mesenchymal precursor cells, or a combination thereof.

17. The pharmaceutical composition of claim 13, wherein the composition is adapted for intra-arterial administration or stereotactic injection.

18. The pharmaceutical composition of claim 13, wherein the composition further comprises a neuroprotective agent.

19. The pharmaceutical composition of claim 13, wherein the composition further comprises a pharmaceutically acceptable carrier.

20. The pharmaceutical composition of claim 13, wherein the pharmaceutically acceptable carrier is pharmaceutically acceptable for use in humans.

21. A method of treating stroke in a subject, the method comprising administering to the subject an effective amount of a composition of claim 13, in a manner in which the composition is delivered to a revascularized bed of tissue in the subject's brain.

22. The method of claim 21, wherein the revascularized bed of tissue is provided by opening an occluded blood vessel in the brain of the subject

23. The method of claim 22, wherein the opening of an occluded blood vessel is accomplished by thrombectomy or thrombolysis.

24. The method of claim 21, wherein the administering of the composition comprises intra-arterial administration or stereotactic injection.

25. The method of claim 21, wherein the intra-arterial administration comprises administration to an internal carotid artery, a vertebral artery, and/or to a branch thereof.

26. The method of claim 21, comprising opening the blood-brain barrier prior to, during, and/or after the administering of the composition.

27. The method of claim 21, wherein opening the blood-brain barrier comprises exposing the subject to focused ultrasound and/or to intra-arterial delivery of mannitol prior to, during, and/or after the administering of the composition.
